SEQUENCE LISTING (2 sequences)

SEQ ID NO: 1 - protein, 7 aa, Unknown
  Description of Unknown Native angiotensin 1-7 peptide sequence
DRVYIHP

SEQ ID NO: 2 - protein, 7 aa, Artificial Sequence
  Description of Artificial Sequence Synthetic peptide
  MOD_RES (1)..(1): Any amino acid or a dicarboxylic acid
  MOD_RES (2)..(2): Arg, Lys, Ala, Cit (citrulline), Orn (ornithine), acetylated Ser, Sar, D-Arg or D-Lys
  MOD_RES (3)..(3): Val, Ala, Leu, Nle (norleucine), Ile, Gly, Lys, Pro, HydroxyPro (hydroxyproline), Aib (2-aminoisobutyric acid), Acpc or Tyr
  MOD_RES (4)..(4): Tyr, Tyr(PO3), Thr, Ser, homoSer (homoserine), azaTyr (aza-alpha1-homo-L-tyrosine) or Ala
  MOD_RES (5)..(5): Ile, Ala, Leu, norLeu, Val or Gly
  MOD_RES (6)..(6): His, Arg or 6-NH2-Phe (6-aminophenylalaine)
  MOD_RES (7)..(7): Cys, Pro or Ala
  See specification as filed for detailed description of substitutions and preferred embodiments
XXXXXXX

(19) United States
(12) Patent Application Publication
(10) Pub. No.: US 2021/0077567 A1
(43) Pub. Date: Mar. 18, 2021

(54) Targeted Angiotensin 1-7 Peptide Conjugates and Formation and Use Thereof

(71) Applicant: IDAHO STATE UNIVERSITY, POCATELLO, ID (US)

(72) Inventor: ALI A. HABASHI, POCATELLO, ID (US)

(21) Appl. No.: 16/937,977

(22) Filed: Jul. 24, 2020

Related U.S. Application Data
(60) Provisional application No. 62/879,780, filed on Jul. 29, 2019.

(51) Int. Cl. A61K 38/08; A61K 47/60; A61K 47/54; A61P 35/00

(52) U.S. Cl. CPC A61K 38/085; A61P 35/00; A61K 47/548; A61K 47/60

(57) ABSTRACT

Angiotensin peptide conjugates, methods of forming the conjugates, and methods of using the conjugates are described. Peptide conjugates include an Ang 1-7-based peptide (e.g., Ang 1-7 or a functional equivalent thereof), a linking agent, and a targeting moiety. Linking agents can include polymeric linkers such as PEG linkers, e.g., monodisperse PEG linkers. Targeting moieties can target tissue or cell types. Targeting moieties can include sulfhydryl groups for targeting hydroxyapatite of bone tissue. Conjugates can exhibit extended plasma half-life and can target bone tissue for use as a reservoir for extended delivery of the peptide.

### CROSS REFERENCE TO RELATED APPLICATION

This application claims filing benefit of U.S. Provisional Patent Application Ser. No. 62/879,780, having a filing date Jul. 29, 2019, which is incorporated herein by reference in its entirety.

### BACKGROUND

The renin-angiotensin system (RAS) is a critical, primary system that regulates multiple vital tissue and organ functions, such as the heart, kidneys and lungs, by maintaining blood pressure homeostasis, electrolyte balance, and inflammatory responses. The RAS is generally viewed as a systemic regulatory mechanism but plays a critical local role in various organs and tissues as well. It has two opposing arms: the ACE/Angiotensin II (AngII)/AT1R classical arm mediating pro-inflammatory, and the alternative ACE2/Angiotensin 1-7 (Ang 1-7)/Mas protective arm. The later counteracts the deleterious actions of AngII imposed through its type1 receptor (AT1R). In a healthy individual, these two arms keep a dynamic balance that maintains tissues and, in general, the body's homeostasis.

The activation of the RAS plays an important role in the pathophysiology of various diseases, and it has been studied as a target for therapeutic intervention in various pathological conditions, including cardiovascular disease, rheumatoid arthritis, and various cancers. Prior to the discovery of ACE2, interventional approaches for targeting the RAS were focused on ACE and Ang II. Such approaches included ACE inhibition (ACEi) and receptor blocking (ARBs) and were focused on attempts to prevent the pro-inflammatory effects of the first arm of the system. More recently, the importance of the second arm in the regulation of the RAS has become evident, and it is believed this arm also presents potential targets for RAS intervention, including the Ang 1-7 peptide. Ang 1-7 has been shown to be a vasodilator agent having functions that are frequently opposed to those attributed to Ang II, which is the major effector component of the RAS. Unfortunately, the limited absorption of Ang 1-7 through biological membranes and very short plasma half-life of the peptide have limited its therapeutic potential.

What is needed in the art are Ang 1-7-based therapeutics that can improve delivery and activity of an Ang-based peptide to encourage beneficial biological actions of the peptide.

### SUMMARY

According to one embodiment, disclosed is a peptide conjugate comprising an Ang peptide, a linking agent bonded to the N-terminus of the Ang peptide at a first end of the linking agent, and a targeting moiety bonded to the linking agent at a second end of the linking agent. In one embodiment, the linking agent can be a polymeric linking agent, e.g., a monodisperse polymeric linking agent such as a monodisperse poly (ethylene glycol) (PEG) linking agent. In one embodiment, the targeting moiety can include a bisphosphonate.

Also disclosed is a method for forming a peptide conjugate. For example, a method can include a solid phase synthesis formation of an Ang peptide during which side chain amines of the Ang peptide are protected. Following, the N-terminus of the peptide can be reacted with a terminus of a linking agent. A method can also include conjugation of a targeting moiety to a second terminus of the linking agent and deprotection of the side chain amines of the peptide to form the peptide conjugate that includes the peptide, the linking agent, and the targeting moiety in a peptide conjugate. The method can form the desired isomer of the peptide conjugate at a high purity (e.g., about 90% purity or greater).

Also disclosed is a method for delivering an Ang peptide to a population of cells that includes contacting the cell population with an Ang conjugate, as described. In one embodiment, the cell population can be a component of bone tissue or in the vicinity of bone tissue or another source of hydroxyapatite. Such methods can be useful in the treatment of joint or bone-related pathologies such as osteoporosis, osteoarthritis or bone metastatic cancers. In other embodiments, the cell population can be independent of hydroxyapatite. Beneficially, the Ang conjugates can exhibit a long half-life. For instance, the Ang conjugates can exhibit a half-life of about 5 hours or more.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 schematically illustrates a native angiotensin 1-7 (Ang 1-7; SEQ ID NO: 1) peptide.

FIG. 2 schematically illustrates one example of an Ang conjugate as described herein incorporating a native Ang 1-7 peptide.

FIG. 3 schematically illustrates a reaction scheme for forming one embodiment of an Ang conjugate as described herein.

FIG. 4 presents the mass spectrometry results of an Ang conjugate.

FIG. 5 presents the HPLC chromatogram results of an Ang conjugate.

FIG. 6 compares cell viability of an SYO1 cancer cell line upon incubation with native Ang II and Ang peptides and with an Ang conjugate as described herein.

FIG. 7 compares cell viability of a Mojo cancer cell line upon incubation with native Ang II and Ang peptides and with an Ang conjugate as described herein.

FIG. 8 compares cell viability of a FUUR1 cancer cell line upon incubation with native Ang II and Ang peptides and with an Ang conjugate as described herein.

FIG. 9 presents results showing tissue distribution of radiolabeled peptides following intravenous (i.v.) or subcutaneous (s.c.) administration of native Ang 1-7 or an Ang conjugate.

FIG. 10 presents the mean plasma activity-time profiles following native Ang 1-7 (i.v.), Ang conjugate (i.v.) and Ang conjugate (s.c.) administration.

FIG. 11 presents the proliferative effects of different concentrations of native Ang II and Ang 1-7 and Ang conjugate on several different cancer cell lines.

FIG. 12 presents the effects of native Ang II and Ang 1-7 and Ang conjugate on the mRNA expression levels of various RAS components in a representative cancer cell line HSSY1.

FIG. 13 presents the effects of native Ang 1-7 and Ang conjugate on tumor size reduction in an osteosarcoma animal model.

FIG. 14 presents the effects of native Ang 1-7 and Ang conjugate on nitric oxide levels in an animal model for prevention of adjuvant induced arthritis in rats. (a, b) represents significant difference between treatments using one-way ANOVA followed by Tukey's adjustment (p<0.05).

FIG. 15 presents the effects of native Ang 1-7 and Ang conjugate on nitric oxide levels in an animal model for treatment of adjuvant induced arthritis in rats. (a, b) represents significant difference between treatments using one-way ANOVA followed by Tukey's adjustment (p<0.05).

FIG. 16 shows relative ACE, ACE 2 and Tubulin expression levels of various treatment groups of adjuvant induced arthritis rats.

FIG. 17 presents the ratio of ACE2 to ACE for various treatment groups of adjuvant induced arthritis rats.

FIG. 18 shows relative MAS and Tubulin expression levels of various treatment groups of adjuvant induced arthritis rats.

FIG. 19 shows the ratio of relative density of MAS to Tubulin for various treatment groups of adjuvant induced arthritis rats.

FIG. 20 illustrates the hydroxyapatite binding of Ang 1-7 and an Ang conjugate in several different mediums.

FIG. 21 presents the tumor size increase for a synovial sarcoma mouse model under various treatment regimens.

FIG. 22 illustrates the effect of various treatments for a synovial sarcoma mouse model on the RAS components gene expression.

FIG. 23 illustrates the effect of various treatments for a synovial sarcoma mouse model on RAS and apoptosis maker protein expression.

FIG. 24 illustrates histology results for control and Ang conjugate treated synovial sarcoma mouse models.

Repeat use of reference characters in the present specification and drawings is intended to represent the same or analogous features or elements of the present invention.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment may be used in another embodiment to yield a still further embodiment.

The present disclosure is generally directed to peptide conjugates based upon Ang peptides, methods of forming the conjugates, and methods of using the conjugates. Ang 1-7 is one of the RAS peptides of the second arm of the RAS. The biological actions of Ang 1-7 include anti-oxidant effects, anti-inflammatory effects, and anti-arrhythmogenic effects, as well as inducing the release of vasodilators. Ang 1-7 also contributes to the beneficial effects of ACE inhibitors and angiotensin II receptor type 1 antagonists. As such, therapeutic agents as described herein can be utilized in one embodiment for the treatment of numerous diseases, including, without limitation, cancer, cardiovascular disease, diabetes, inflammation, viral infection, pulmonary diseases, Parkinson's disease, and Alzheimer's disease.

Disclosed conjugates are directed in one embodiment to peptide therapies that can directly or indirectly utilize the activity of an Ang 1-7-based peptide to signal cells of a cell population to stop growing and die. This approach is particularly feasible for use with an Ang 1-7-based peptide because the Ang 1-7 of the RAS is a relatively small peptide of 7 amino acids and, as such, is typical of peptides that control the balance of cellular growth, which are generally in the range of 5-12 amino acids. This relatively small size makes the peptide more readily modified by formation of a conjugate that can exhibit desired solubility, distribution, and/or other pharmacokinetic properties. Disclosed Ang conjugates provide chemical stabilization of the peptide through formation of a conjugate, and this chemical stabilization can increase the plasma half-life of the peptides, as well as improve absorption of the peptides.

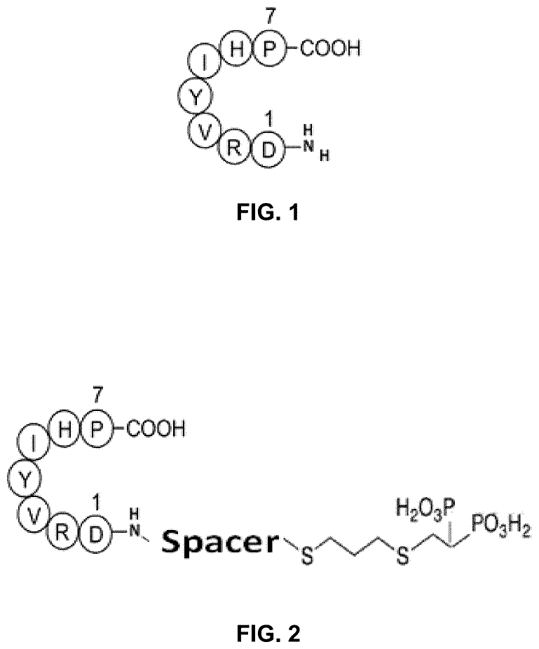

In one embodiment, the peptide of the conjugate can include a native Ang 1-7 peptide, as schematically illustrated in FIG. 1 (SEQ ID NO: 1). However, a peptide conjugate is not limited to inclusion of the native Ang 1-7 peptide, and in other embodiments, an analogue or functional fragment of a native Ang 1-7 peptide can be utilized. As utilized herein, the terms “Ang peptide” and “Ang 1-7-based peptide” refer to the native Ang 1-7 peptide, as well as any peptide analogue (either peptidic or non-peptidic) or functional fragment of an Ang peptide or analogue.

Ang peptides, both peptidic and non-peptidic, have been described and are known in the art. For example, Ang 1-7 analogues as may be incorporated in a peptide conjugate as described herein have been described in U.S. Pat. No. 8,835,375 to Haas, et al.; U.S. Patent Application Publication No. 2014/0031286 to Rodgers, et al.; and U.S. Pat. No. 9,333,233 to Franklin, all of which are incorporated herein by reference for all purposes.

In some embodiments, a functional equivalent, analogue or derivative Ang peptide can be a fragment of the native Ang-1-7 (SEQ ID NO: 1) of FIG. 1 or can include amino acid substitutions, deletions and/or insertions in the native Ang-1-7 (SEQ ID NO: 1) of FIG. 1. Ang peptide functional equivalents, analogues or derivatives can be made by altering the native amino acid sequences by substitutions, additions, and/or deletions. For example, one or more amino acid residues within the sequence of the native Ang-1-7 (SEQ ID NO: 1) can be substituted by another amino acid of a similar polarity, which acts as a functional equivalent, resulting in a silent alteration. Substitution for an amino acid within the sequence may be selected from other members of the class to which the amino acid belongs. For example, the positively charged (basic) amino acids include arginine, lysine, and histidine. The nonpolar (hydrophobic) amino acids include leucine, isoleucine, alanine, phenylalanine, valine, proline, tryptophan, and methionine. The uncharged polar amino acids include serine, threonine, cysteine, tyrosine, asparagine, and glutamine. The negatively charged (acid) amino acids include glutamic acid and aspartic acid. The amino acid glycine may be included in either the nonpolar amino acid family or the uncharged (neutral) polar amino acid family. Substitutions made within a family of amino acids are generally understood to be conservative substitutions.

An Ang peptide can be of any length and is not limited to 7 amino acids as in the native peptide. In some embodiments, an Ang peptide of the conjugate can contain, for example, from 4-25 amino acids (e.g., 4-20, 4-15, 4-14, 4-13, 4-12, 4-11, 4-10, 4-9, 4-8, 4-7 amino acids). In some embodiments, the peptide contains 4, 5, 6, 7, 8, 9, 10, 11, 12, 13, 14, 16, 17, 18, 19, 20, 21, 22, 23, 24, or 25 amino acids.

The Ang peptides and peptide analogs can be represented in one embodiment by the following sequence:

Xaa¹-Xaa²-Xaa³-Xaa⁴-Xaa⁵-Xaa⁶-Xaa⁷ (SEQ ID NO: 2), or a pharmaceutically acceptable salt thereof in which:

Xaa¹ can be any amino acid or a dicarboxylic acid. In some embodiments, Xaa¹ can be Asp, Glu, Asn, Acpc (1-aminocyclopentane carboxylic acid), Ala, Me₂Gly (N,N-dimethylglycine), Pro, Bet (betaine, 1-carboxy-N,N,N-trimethylmethanaminium hydroxide), Glu, Gly, Asp, Sar (sarcosine) or Suc (succinic acid). In one embodiment, Xaa¹ can be a negatively-charged amino acid, such as Asp or Glu.

Xaa² can be Arg, Lys, Ala, Cit (citrulline), Orn (ornithine), acetylated Ser, Sar, D-Arg or D-Lys. In some embodiments, Xaa² can be a positively-charged amino acid, such as Arg or Lys.

Xaa³ can be Val, Ala, Leu, Nle (norleucine), Ile, Gly, Lys, Pro, HydroxyPro (hydroxyproline), Aib (2-aminoisobutyric acid), Acpc or Tyr. In some embodiments, Xaa³ can be an aliphatic amino acid, such as Val, Leu, Ile or Nle.

Xaa⁴ can be Tyr, Tyr(PO₃), Thr, Ser, homoSer (homoserine), azaTyr (aza-al-homo-L-tyrosine) or Ala. In some embodiments, Xaa⁴ can be a hydroxyl-substituted amino acid such as Tyr, Ser or Thr.

Xaa⁵ can be Ile, Ala, Leu, norLeu, Val or Gly. In some embodiments, Xaa⁵ can be an aliphatic amino acid, such as Val, Leu, Ile or Nle.

Xaa⁶ can be His, Arg or 6-NH₂-Phe (6-aminophenylalaine). In some embodiments, Xaa⁶ can be a fully or partially positively-charged amino acid, such as Arg or His.

Xaa⁷ can be Cys, Pro or Ala.

In some embodiments, one or more of Xaa¹-Xaa⁷ can be identical to the corresponding amino acid in native Ang 1-7 (SEQ ID NO: 1). In one embodiment, all but one or two of Xaa¹-Xaa⁷ can be identical to the corresponding amino acid in native Ang 1-7 (SEQ ID NO: 1). In other embodiments, all of Xaa¹-Xaa⁶ can be identical to the corresponding amino acid in native Ang 1-7 (SEQ ID NO: 1).

In one embodiment, the Ang peptide of a conjugate can exhibit 50, 55, 60, 65, 70, 75, 80, 85, 90, 95, 96, 97, 98, or 99% homology to the reference sequence of the native Ang 1-7 peptide (SEQ ID: 1).

An Ang peptide may be obtained by any method known to those skilled in the art, including synthetic and recombinant techniques. By way of example, synthetic formation techniques including, without limitation, exclusive solid phase synthesis, partial solid phase synthesis, fragment condensation, classical solution synthesis, or native-chemical ligation can be utilized.

In one embodiment, an Ang peptide can be obtained by solid phase peptide synthesis as is generally known in the art. Briefly, a solid phase synthesis method can include coupling the carboxyl group of the C-terminal amino acid of the peptide to a suitable resin (e.g., benzhydrylamine resin, chloromethylated resin, hydroxymethyl resin) and successively adding N-alpha protected amino acids. The protecting groups may be any such groups known in the art. Two common N-terminal protecting groups as may be utilized are tert-butoxycarbonyl (Boc) and 9-fluorenylmethoxycarbonyl (Fmoc). In one embodiment, the protecting groups can remain on the peptide through formation, so as to prevent reaction with other components of the conjugate during formation of the peptide conjugate.

Solid phase synthesis has been disclosed, for example, by Merrifield, J. Am. Chem. Soc. 85: 2149 (1964); Vale et al., Science 213:1394-1397 (1981); in U.S. Pat. No. 4,305,872 to Johnston, et al.; and U.S. Pat. No. 4,316,891 to Guillemin, et al. (both of which are incorporated herein by reference for all purposes); Bodonsky et al. Chem. Ind. (London), 38:1597 (1966); and Pietta and Marshall, Chem. Comm. 650 (1970) by techniques reviewed in Lubell et al. “Peptides” Science of Synthesis 21.11, Chemistry of Amides. Thieme, Stuttgart, 713-809 (2005). The coupling of amino acids to appropriate resins is also well known in the art and has been disclosed, for instance in U.S. Pat. No. 4,244,946 to Rivier, et al., which is incorporated herein by reference for all purposes.

One embodiment of an Ang peptide conjugate is illustrated in FIG. 2. As shown, the peptide conjugate can include a spacer that can be a polymeric or non-polymeric linking agent conjugated to the Ang peptide. In one embodiment, the spacer (alternately referred to throughout this disclosure as a linking agent) can be conjugated to the peptide in such a fashion so as to maintain the activity of the peptide. For instance, the linking agent can be bonded to the peptide only at the N-terminus of the Ang peptide.

In one embodiment, the linking agent can include a linear or branched water-soluble and non-peptide polymer. In some embodiments, the linking agent can be one or more of soluble in water, stable to heat, inert to many chemical agents, resistant to hydrolysis, and nontoxic. The linking agent can be biocompatible, and as such, can be capable of coexistence with living tissues or organisms. The linking agent can be non-immunogenic, and as such, is not known to produce an immune response in the body.

A polymeric linking agent can encompass any polymer that includes one or more reactive functional groups that allow for covalent bonding with an Ang peptide and a targeting moiety, e.g., a bisphosphonate or bisphosphonate-containing group. In one embodiment, the polymeric linking agent can include a poly (alkylene glycol), e.g., a linear poly (alkylene glycol) such as a linear poly (ethylene glycol) (PEG). A polymeric linking agent is not limited to PEG-based polymers, however, and in other embodiments, polymeric linking agents as are known in the art may alternatively be utilized, examples of which include, without limitation, dextran, water soluble polyamino acids, polyglutamic acid (PGA), polylactic acid (PLA), polylactic-co-glycolic (PLGA), poly(D,L-lactide-co-glycolide) (PLA/PLGA), poly (hydroxy alkyl methacrylamide), polyglycerol, poly (amidoamine) (PAMAM), and polyethylenimine (PEI).

A polymeric linking agent is not particularly limited with regard to size. For instance, suitable PEG-based linking agents can include, without limitation, PEG(100), PEG(200), PEG(300), PEG(400), PEG (500), PEG(600), PEG(1000), PEG(1500), PEG(2000), PEG(3000), PEG(3350), PEG(4000), PEG(5000), PEG(6000), PEG(8000), and PEG(10000), as well as methoxy and ethoxy derivatives thereof, and any PEG having a molecular size within and inclusive of any of the above indicated molecular weights, as well as larger or smaller polymers.

A linking agent is not limited to polymeric linking agent. For instance, a non-polymeric crosslinking agent can be incorporated in an Ang peptide conjugate as a linking agent between the Ang peptide and the targeting moiety.

By way of example, exemplary linking agents can include sulfosuccinimidyl-4-(N-maleimidomethyl)cyclohexane-1-carboxylate (Sulfo-SMCC); m-maleimidobenzoyl-N-hydroxysulfosuccinimide ester (Sulfo-MBS); 3-Maleimidobenzoyl-N-hydroxysuccinimide ester (MBS); sulfosuccinimidyl 6-(3′-[2-pyridyldithio]-propionamido)hexanoate (Sulfo-LC-SPDP); N-succinimidyl(4-iodoacetyl)aminobenzoate (SIAB); maleimido butryloxy-succinimide ester (GMBS); N-(e-MaleimidoCaproyloxy)-N-HydroxySuccinimide ester (EMCS); succinimidyl-6-((iodoacetyl)amino)hexanoate (SIAX); Succinimidyl-4-(p-maleimidophenyl)butyrate (SMPB); succinimidyl-4-(((iodoacetyl)amino)methyl) cyclohexane-1-carboxylate (SIAC); p-nitrophenyl iodoacetate (NPIA), m-maleimidobenzoyl-N-hydroxysulfosuccinimide ester (Sulfo-MBS); N-(e-MaleimidoCaproyloxy)-N-HydroxySuccinimide ester (EMCS); p-nitrophenyl iodoacetate (NPIA); as well as derivatives thereof (e.g., Sulfo-SIAB, Sulfo-GMBS, Sulfo-EMCS, etc.), or any combination thereof.

In one embodiment, characteristics of the linking agent, e.g., the weight average and/or number average molecular weight of a polymer, etc., can be modified to control characteristics of the conjugate, as well as characteristics of the activity of the Ang peptide carried by the conjugate. For instance, modification of the length of a linear polymeric linking agent can be utilized to effect half-life of the conjugate and/or release profile of the peptide.

In one embodiment, the conjugate can incorporate a linking agent having a well-defined structure, with little or no variations between individual members (e.g., a single isomer), which can provide a route for formation of a well-defined conjugate at high purity, and thus, provide for tight control capability of the characteristics of the conjugate. In one embodiment, the linking agent can incorporate a polymer having a narrow molecular weight distribution, and in one embodiment, a monodisperse polymer, e.g., a monodisperse PEG, such as a monodisperse linear PEG.

In one embodiment, a narrow molecular weight polymeric spacer can be formed from free radical polymerization followed by separation via, e.g., size exclusion chromatography. In free radical polymerization processes, molecular weight distributions can be narrowly controlled for chains having molecular weights between about 200 and 1,200 daltons and above. Typically, far less than 50% of the polymers in a formation batch have exactly the targeted molecular weight. Narrower-distribution may be achieved with size exclusion chromatography, which can result in a greater amount of the polymer, e.g., about 80% or more of PEG polymers having a targeted molecular weight.

In one embodiment, a monodisperse polymer can be utilized as a polymeric linking agent. For instance, monodisperse PEG containing up to about 50 ethylene oxide units is available in the retail market, including linear PEG, as well as branched structures that include from about 3 to about 9 monodisperse linear chains. In one embodiment, the polymeric linking agent can include a linear monodisperse PEG of from about 3 to about 48 ethylene oxide units, from about 5 to about 40 ethylene oxide units, from about 10 to about 35 ethylene oxide units, or from about 15 to about 30 ethylene oxide units, in some embodiments. For instance, a polymeric linking agent can include 2, 4, 8, 10, 12, 15, 17, 19, 20, 23 25, 27, 30, 35, 40, or 45 ethylene oxide units, in some embodiments.

A polymeric linking agent can include one or more reactive functional groups that allow for covalent bonding with the Ang peptide and a targeting moiety. In one embodiment, the polymeric linker can include exhibit heterofunctionality, e.g., two different reactive functional groups—one designed for covalent bonding with the N-terminus of the Ang peptide and one designed for covalent bonding with the targeting moiety—but this is not a requirement of the invention, and in other embodiments, the polymeric linker can include the same reactive functionality for bonding to both components. Suitable bonding functionalities are not particularly limited, examples of which can include, without limitation, hydroxyl, active ester; active carbonate; acetal; aldehyde; aldehyde hydrate; alkyl or aryl sulfonate; halide; disulfide; alkenyl; acrylate; methacrylate; acrylamide; active sulfone; amine; hydrazide; thiol; carboxylic acid; isocyanate; isothiocyanate; maleimide; vinylsulfone; dithiopyridine; vinylpyridine; iodoacetamide; epoxide; glyoxal; dione; mesylate; tosylate; or tresylate. Reactive functionality can be incorporated by use of a biologically-based functional group, e.g., biotin functionality or the like.

Conjugation between the Ang peptide and the linking agent can be carried out according to any suitable chemistry, which can usually depend upon the particular functional groups utilized in the conjugation reaction, as is known to one of ordinary skill in the art. In those embodiments in which the N-alpha amino acids of the peptide are protected, the conjugation reaction can form exclusively (or nearly exclusively, e.g., about 90% or greater) isomers in which the linking agent is bonded only to the N-terminus of the Ang peptide.

At a second end of the linking agent, the Ang conjugate can include at least one targeting moiety. In one embodiment, the targeting moiety can be a bone targeting moiety that includes a thiol functional group (—SH). However, the targeting moiety is not limited to bone targeting, and the Ang conjugates can include targeting moieties directed toward other tissues than bone, as well as particular cell types, ECM components, etc. For example, on a second end of spacer, an Ang peptide conjugate can include a folate molecule (vitamin B9) that can target cancer cells expressing folate receptor, or a small peptide binding element as is known in the art, e.g., Arg-Gly-Asp (RGD) as can be utilized to target αvβ3 motif of integrin, etc.

In one embodiment, the targeting moiety can include a thiol-containing component for targeting a component of bone tissue. In one particular embodiment, a bone targeting moiety can include a bisphosphonate component, one exemplary embodiment being illustrated in the conjugate of FIG. 2. As utilized herein, the term “bisphosphonate” includes any salts, solvates and/or hydrates of bisphosphonate structures as defined herein. For instance, a bone targeting moiety can be a bisphosphonate having the following general structure:

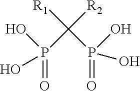

in which at least one of R₁ and R₂ includes a functional group that allows for covalent bonding with the polymeric linking agent. For example, R₁ and R₂ can be independently selected from hydrogen, substituted or unsubstituted alkyl, substituted or unsubstituted cycloalkyl, substituted or unsubstituted aryl, substituted or unsubstituted heteroaryl, substituted or unsubstituted heteroalicyclic, halo, hydroxy, thiol, alkoxy, thioalkoxy, aryloxy, thioaryloxy, amine, or alkylamine.

In some embodiments, at least one of R₁ and R₂ is hydroxy or hydrogen, and the other one can include a thioalkyl, alkylamine, or alkoxy. An alkyl chain of a bisphosphonate can be varied, variation of which can, in one embodiment, be utilized to modify and design desirable characteristics for a conjugate. For instance, in some embodiments, an alkyl chain of a bisphosphonate can have from 1 to about 10 carbon atoms in the backbone chain, or from 1 to about 6, or from about 2 to about 5 carbon atoms in some embodiments.

By way of example, in one embodiment, a bisphosphonate for inclusion on a peptide conjugate can have a structure of:

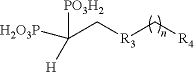

in which n is from 1 to 10, R₃ is selected from S, N, O, and R₄ includes a functional group that allows for covalent bonding with the polymeric linking agent, examples of which can include, without limitation, hydroxyl; active ester; active carbonate; acetal; aldehyde; aldehyde hydrate; alkyl or aryl sulfonate; halide; disulfide; alkenyl; acrylate; methacrylate; acrylamide; active sulfone; amine; hydrazide; thiol; carboxylic acid; isocyanate; isothiocyanate; maleimide; vinylsulfone; dithiopyridine; vinylpyridine; iodoacetamide; epoxide; glyoxal; dione; mesylate; tosylate; or tresylate.

Bisphosphonates are widely used for the treatment of osteoporosis and have also been used as a vehicle for delivering bone-targeted prodrugs to osseous tissue via the bisphosphonic moiety that binds hydroxyapatite. The bisphosphonate component of the Ang conjugates described herein can thus be utilized in one embodiment for targeting bone tissue for therapeutic purposes or for utilizing bone tissue as a reservoir for sustained release of the peptides. As such, in some embodiments, the Ang peptide conjugates can be utilized in therapies directed to bone or joint tissue.

By way of example, disclosed Ang peptide conjugates can be utilized in treatment or study of osteosarcoma as the bisphosphonate component of the conjugate can assist in homing the peptide to bone tissue where the cancer is located. In another embodiment, disclosed conjugates can be utilized in treatment or study of rheumatoid arthritis (RA), which is a chronic inflammatory condition that plagues 1% of the population and is known to activate the RAS. The activated RAS is the main contributing factor in causing cardiovascular mortality of RA patients, due to the augmentation of Ang II local and systemic levels of Ang II. Delivery of an Ang peptide via disclosed conjugates can thus be utilized to counteract the cardiotoxic effects of Ang II.

Conjugation between the targeting moiety and the linking agent can be carried out according to any suitable chemistry, which can usually depend upon the particular functional groups utilized in the conjugation reaction, as is known to one of ordinary skill in the art.

As discussed previously, in one embodiment, an Ang peptide can be formed according to a solid phase peptide synthesis in which the N-alpha protecting groups can remain on the peptide throughout formation of the conjugate. In this embodiment, the protecting groups can be removed following formation of the conjugate, or alternatively, following conjugation between the linking agent and the conjugate and prior to conjugation with the bisphosphonate component. Deprotection of the protecting groups can be carried out according to any suitable methodology; for instance, by use of a moderately strong acid (e.g., trifluoroacetic acid) when utilizing Boc as a protecting group or by use of a mild base (e.g., piperidine) when utilizing Fmoc as a protecting group.

Formation methods as may be utilized in preparing the disclosed conjugates can provide an Ang peptide conjugate at high purity. For instance, through utilization of solid phase synthesis peptide formation techniques that maintain the N-alpha protecting groups during formation of the conjugate, the conjugation reaction between the Ang peptide and the linking compound can be targeted to only the N-terminus of the peptide, and formation of multiple isomers can be prevented. Moreover, through utilization of a linking agent having a single isomer and/or a narrow molecular weight range, or, in one embodiment, a monodisperse polymeric linking agent, the Ang peptide conjugate structure can be even more narrowly controlled, and as such, can exhibit well-defined characteristics. For instance, in one embodiment, a formation method can provide a monodisperse single isomer of the Ang peptide conjugate at a high purity of about 90% or higher.

The conjugation of an Ang peptide with a targeting moiety can be utilized to target the Ang peptide to tissue and utilize the tissue as a reservoir for sustaining therapeutic plasma levels of the active peptide in the targeted area. Moreover, the conjugate can provide for high stability in solution and prolongation of plasma half-life of the Ang peptide conjugate as compared to the native peptide. For instance, an Ang peptide conjugate as described herein can exhibit a plasma half-life of about 60 minutes or more, about 120 minutes or more, about 200 minutes or more, about 300 minutes or more, or about 400 minutes or more, in some embodiments. For example, an Ang peptide conjugate can exhibit a 10-fold increase in plasma half-life after intravenous administration and a 15-fold increase in half-life after subcutaneous injection as compared to the native peptide, which can result in longer mean resistance time for the Ang peptide of the conjugate compared with that of the native peptide alone.

The present disclosure may be better understood with reference to the Examples set forth below.

### EXAMPLE 1

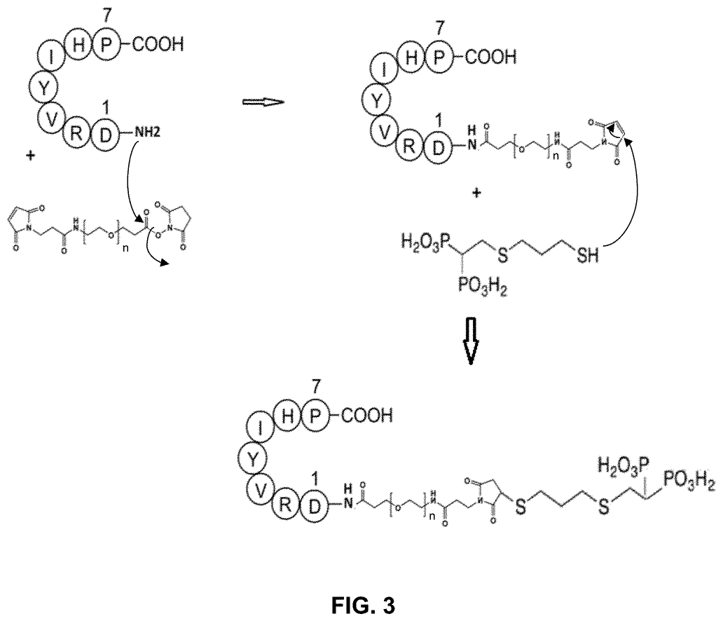

Conjugates were prepared by PEGylating of an Ang peptide with a spacer followed by conjugation with a thiol-bisphosphonate as illustrated in FIG. 3 using a Fluorenylmethyloxycarbonyl (Fmoc)/Tertiary butyl (tBu) solid phase synthesis method. The peptides were produced by starting with a single amino acid and elongating one by one based on the sequence order using solid phase synthesis to produce Ang 1-7. To prepare the conjugates, potential unwanted reaction sites of the peptides (e.g., amines of side chains in arginine and histidine) were protected from reaction by use of the solid phase synthesis process in which amine groups of the side chains were protected with a protecting group that was not cleaved from the peptide at formation. Following formation of the peptide, 200 μL of Ang peptide solution (10 mg/mL in DMSO) was mixed with 100 μL of spacer compound (a 27-mer mono-disperse PEG(maleimide)₂) (50 mg/mL, in DMSO) in room temperature for 1 hour while rotating gently. In a second step, to the mixer was added 2 mL of Thiol-BP solution (25 mg/mL in phosphate buffer, 100 mM, pH 7.4) to form a conjugate, as illustrated in FIG. 3. Following completion of the reaction, the protected amino acids residues of the Ang peptide were deprotected. Tert-butyloxycarbonyl (BOC] protected side chain was be deprotected using treatment with mixture of TFA/DCM at RT for 2 hours, after which time the volatiles were removed by vacuum.

The final solution was dialyzed to remove unreacted components. After confirmation of the conjugate formation using mass spectrophotometer, the resulting conjugate was separated using an analytical reverse-phase HPLC with UV-VIS detector, and eluted major peaks were collected using fraction collector and freeze-dried overnight. The resulting powders were submitted to mass spectroscopy to confirm their molecular mass.

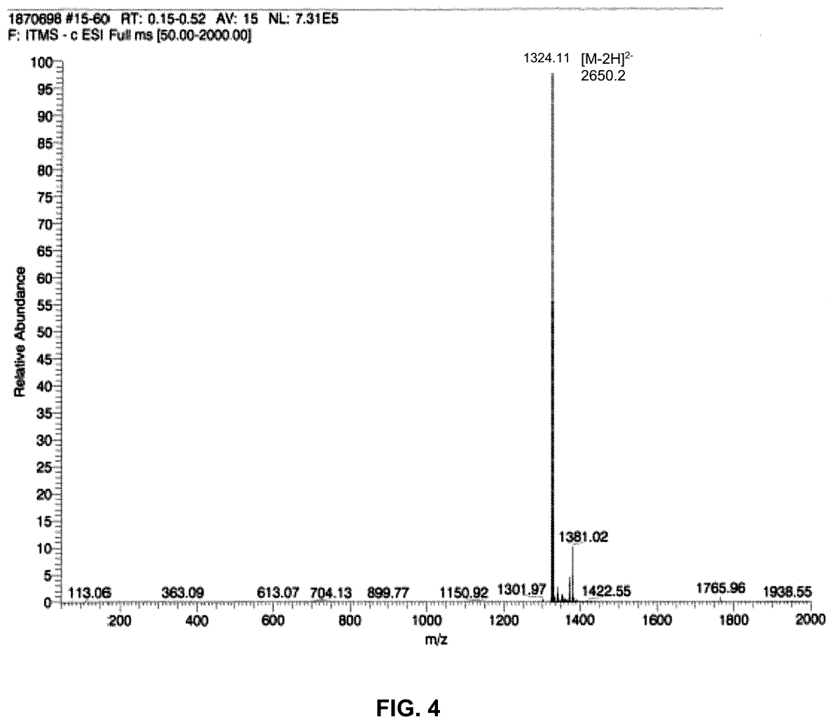

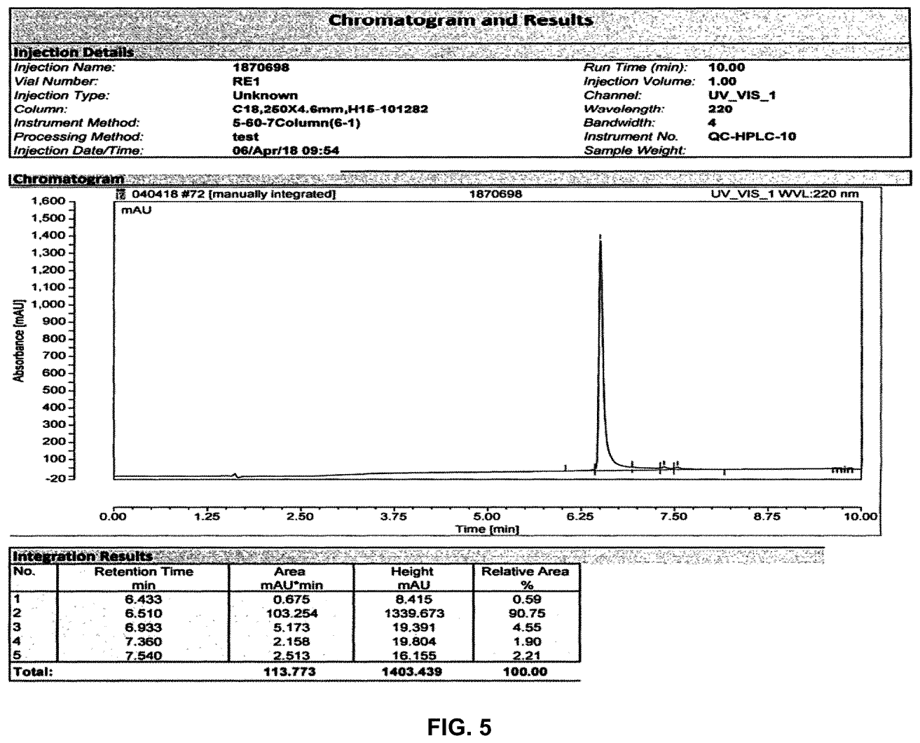

As indicated in FIG. 4 (mass spectroscopy results) and FIG. 5 (HPLC chromatogram results), the formation strategy accomplished formation of the targeted site conjugation. The use of a monodisperse linking agent and the nature of conjugation between the peptide and the linker (i.e., a single conjugation reaction between the peptide and the linking agent) significantly reduced the number of potential conjugation products and simplified the identification of the successfully formed conjugate. Results indicated formation of a conjugate having a molecular weight of 2650.8 g/mol and a purity of greater than 90.75%.

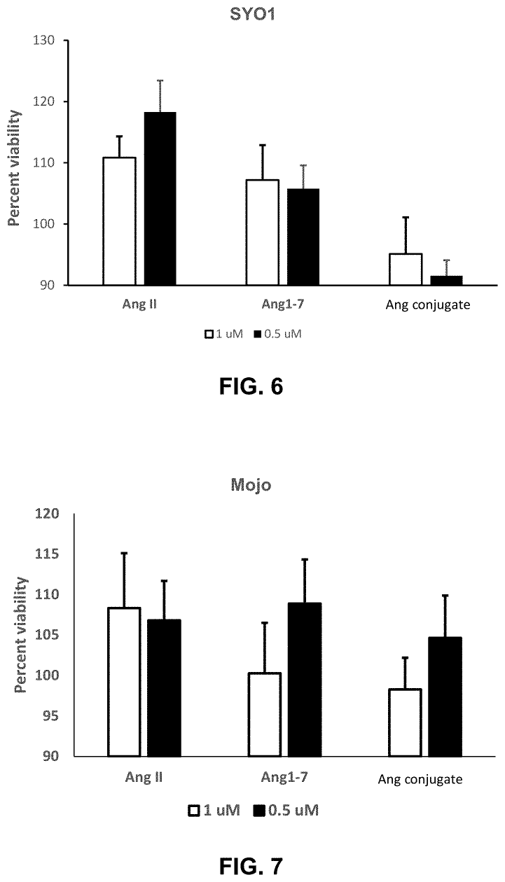

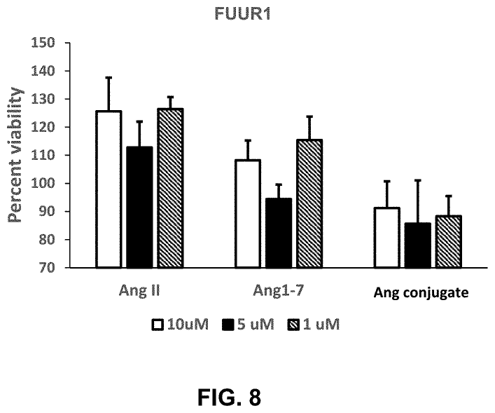

Upon confirmation of final chemical structure of the active conjugates, the anti-proliferative activity was tested using different cancer cell lines, including synovial sarcoma cell lines SYO1 and MoJo, and the renal carcinoma cell line FUUR1. Results comparing the percent viability of the tested cell lines upon incubation with AngII, Ang1-7, or the Ang peptide conjugate are provided in FIG. 6 (SYO1), FIG. 7 (MoJo) and FIG. 8 (FUUR1). The cell lines were incubated with concentrations of the tested materials at various concentrations, as shown. The results of the in vitro cell viability tests indicate superior activity for the conjugates as compared with native peptides.

Pharmacokinetic studies were conducted using rats for calculation of biological half-life and other pharmacokinetic parameters of the conjugates in comparison with native peptides. The tissue distribution of conjugates was conducted on main organs, such as bone (major target), heart, liver, kidney, lung, etc.

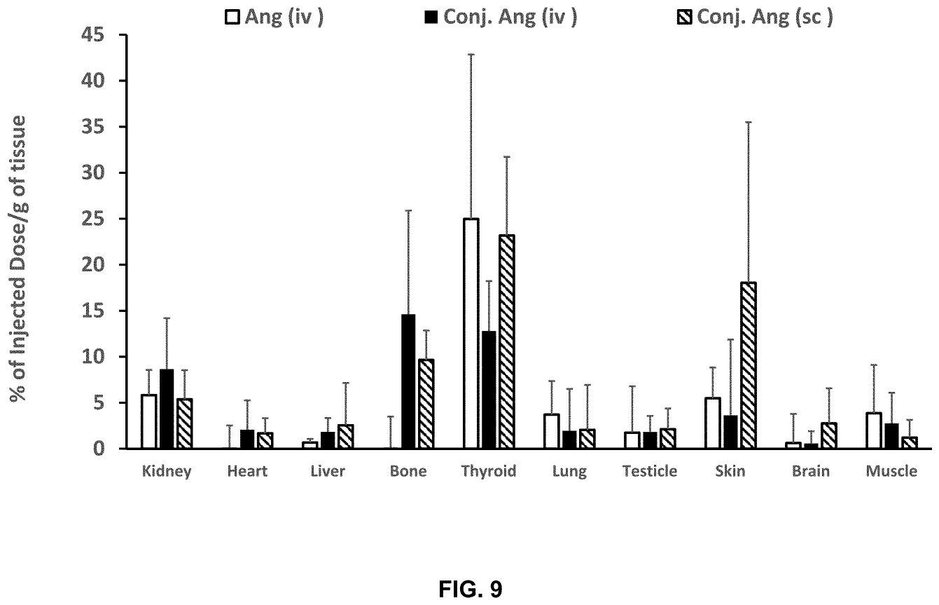

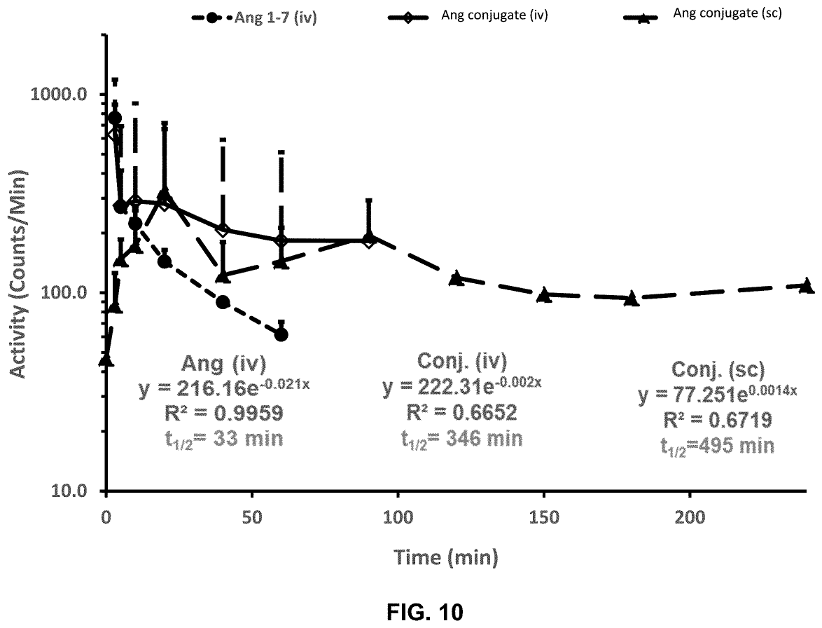

As expected, the conjugates demonstrated higher affinity to thyroid and bone (FIG. 9) and longer half-life (FIG. 10), as compared to the native peptides. Conjugates formed as described in this example were used in the following examples.

### EXAMPLE 2

The viability of several different cancer cell lines including FUUR1, MoJo, SYO1, and the sarcoma cell line HSSY1 using the MTT assay across multiple concentrations and time points and compared with native Ang 1-7 as positive control and Ang II as negative control. The impact of Mas receptor activation by Ang peptide conjugate on gene expression of different components of the RAS, such as ACE1, ACE2, AT1R. Mas receptor was also studied using quantitative PCR using the different cancerous cell lines.

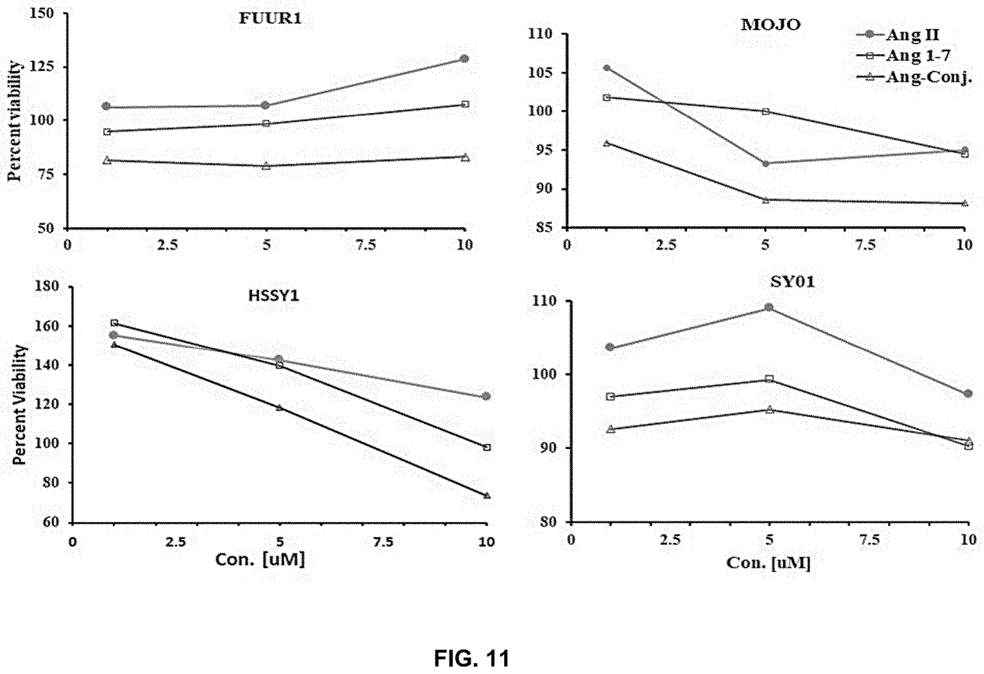

FIG. 11 illustrates the results. As shown, Ang II demonstrates a remarkable proliferative effect in all cell lines tested. In contrast, treatment with native Ang 1-7 resulted in significant concentration dependent decrease on cell proliferation. However, the anti-proliferative effect was more pronounced in the Ang peptide conjugate treated cells.

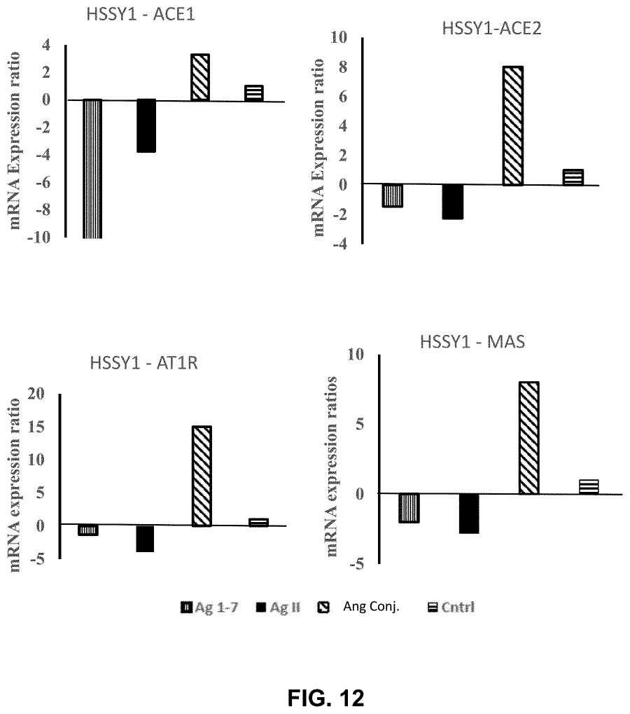

As indicated in FIG. 12, gene expression of the RAS components after different treatments was variable. Although the gene expression of some of the components from classical RAS, such as ACE and AT1R, was enhanced to some level after Ang peptide conjugate treatment, the elements of protective arm such as ACE2 and Mas receptor expression ratio were significantly higher, as well. As indicated, native Ang 1-7 did not significantly change the gene expression of ACE2, AT1R and Mas, but it had very large effect on ACE gene expression.

### EXAMPLE 3

The Ang peptide conjugate described in Example 1 was examined for inhibition of the RAS in an advanced form of osteosarcoma. For the in vivo study, a group of mice bearing osteosarcoma tumors was divided into three different groups and subcutaneously dosed twice daily with vehicle (PEG 400: normal Saline, 3:2), Ang 1-7, or Ang peptide conjugate (equivalent dose of Ang 1-7 of 400 μg/kg) for three weeks. The tumor size was measured three times a week. Tumor tissues were harvested and kept in appropriate medium in −80° C. for further analysis of different cancer biomarkers.

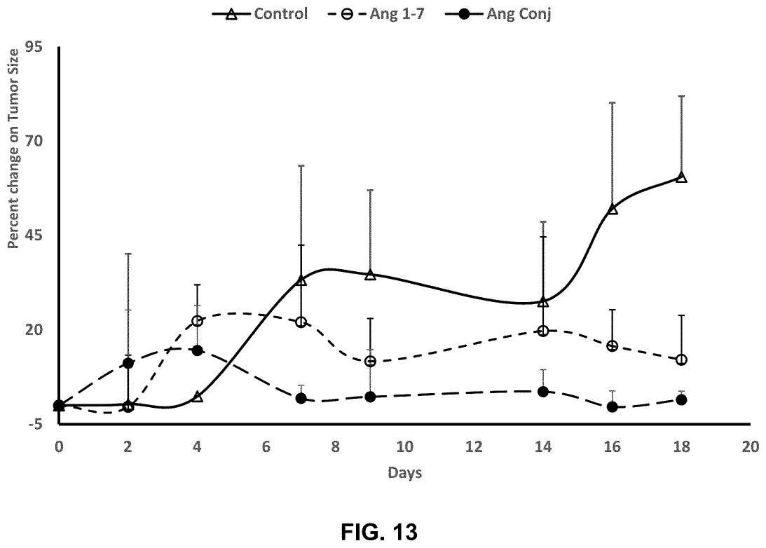

Results are illustrated in FIG. 13. As shown, Ang peptide conjugate potently inhibited tumor growth after 3-4 days, while Ang 1-7 had milder effect after 10 days.

### EXAMPLE 4

The Ang peptide conjugate described in Example 1 was examined for anti-inflammatory effect on adjuvant-induced arthritis in a rat animal model.

On day one, for induction of adjuvant arthritis, male Sprague Dawley rats were injected at the tail base with 0.2 mL of 50 mg/mL Mycobacterium butyricum. Arthritic animals were divided into 4 groups and treated with saline, Ang II, Ang 1-7 or Ang peptide conjugate. A group of healthy rats received vehicle as control group. Preventive and treatment groups subcutaneously received 200 μg/kg equitant dose daily from day one for 3 weeks or after emergence of arthritis for 1 week, respectively. Animals were monitored daily for their body weight, paw and joint diameters. Animals were then euthanized, plasma/tissues were harvested and analyzed for plasma nitric oxide (NO), and the RAS components.

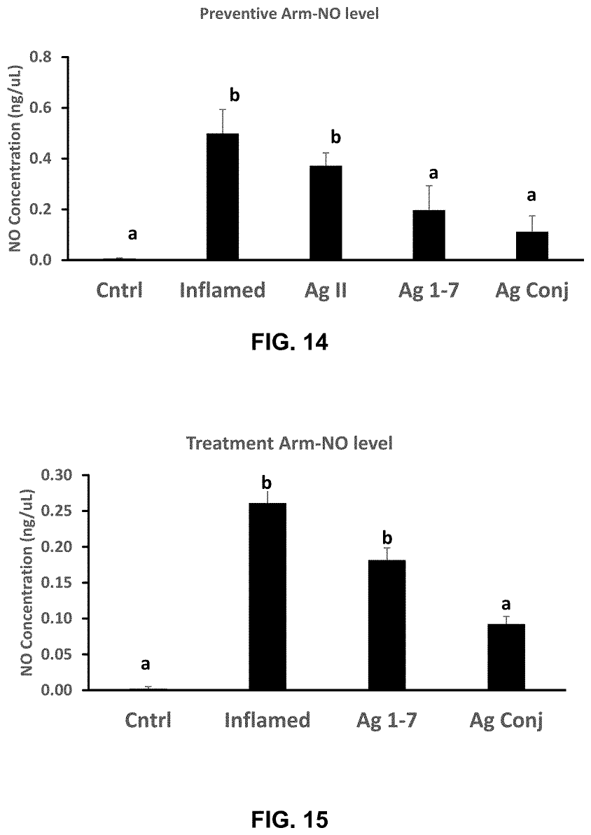

NO is a biomarker of inflammation, which has been shown to be elevated in AA rats compared with control animals in both arms of study. NO concentration is more pronounced in preventive arm then the treatment arm as symptoms of AA decline over time do to natural resolution of inflammation. Results for the preventative group are provided in FIG. 14, and results for the treatment group are provided in FIG. 15. As can be seen, the results indicate that the Ang peptide conjugate was able to reduce the elevated plasma level of NO in both the preventive and treatment approach. This was in concert with visual signs of inflammation presented as reduced body weight gain and swelling of paw and joints of arthritic animals.

Additional results are shown in Tables 1 and 2 below. As shown, treatment with the Ang conjugate reduced the AA induced elevated level of NO in both the preventive arm and the treatment arm. As shown in Table 1, below, arthritic animals had lower weight gain than those in the control group, and animals treated with Ang conjugate showed improvement in that respect to a greater extent than animals treated with the native peptide (Inf.). In the figure, * indicates significant difference from the control group (p<0.05), as well as the inflamed group (p<0.05)

TABLE 1

| | Mean body | Difference vs. |
|---|---|---|
| Group | weight (SD) | Inf- Vehicle |
| Daily dose of Ang Conj. restores body weight gain | | |
| (%) compared with day 1 in preventive arm | | |
| Control | 21.2 (2.6) * | 12.29 |
| Inf-Vehicle | 8.9 (6.0) | |
| Inf-Ang II | 13.2 (3.1) | 4.32 |
| Inf-Ang 1-7 | 13.6 (8.8) | 4.78 |
| Inf-Ang Conj. | 15.1 (12.0) | 6.22 |
| Daily dose of Ang Conj. restores body weight gain | | |
| (%) after 7 days of dosing in treatment arm | | |
| Control | 5.7 (1.3) | 1.5 |
| Inf-Vehicle | 4.2 (1.5) | |
| Inf-Ang 1-7 | 10.0 (4.8) * | 5.7 |
| Inf-Ang Conj. | 7.3 (4.3) * | 3.0 |

* Significantly different from Inf-Vehicle, p < 0.05
Inf - inflamed animal

Table 2, below, presents the percent change in paw and joint diameters, which increased due to swelling and inflammation in arthritic animals. As indicated, Ang II, as a pro-inflammatory agent, made the situation worse, but treatment with Ang 1-7, and to a higher extent Ang conjugate, reduced the inflammation and swelling to a comparable level with the control group in both the preventive and treatment groups of the study.

TABLE 2

| | Paw Diameter (mm) | | Joint Diameter (mm) | |
|---|---|---|---|---|
| Group | L. Hind | R. Hind | L. Hind | R. Hind |

Daily dose of Ang Conj. reduces (% change) AA induced
swelling of paws and joints compared with Day 1 in
preventive arm

| | | | | |
|---|---|---|---|---|
| Control | −1.5(5.9)* | 0.2(4.8)* | 4.0(4.7) | 3.6(3.6)* |
| Inf-Vehicle | 13.0 (8.5) | 8.5(9.8) | 9.7(4.3) | 11.0(3.1) |
| Inf-Ang II | 15.9(5.8) | 20.3(4.0) | 5.4(3.0) | 15.1(2.9) |
| Inf-Ang | 0.5 (4.8)* | 0.2(2.4)* | −0.7(6.2)* | 3.1(3.6)* |
| 1-7 | | | | |
| Inf-Ang | −1.1 (6.6)* | −3.4(1.6)* | 5.4(1.6)* | 1.0(3.8)* |
| Conj. | | | | |

Daily dose of Ang Conj. reduces (% change) AA induced
swelling of paws and joints after 7 days of dosing in
treatment arm

| | | | | |
|---|---|---|---|---|
| Control | 1.1(1.0) * | 1.9(3.6) * | 0.4(2.7) * | 2.0(1.5) * |
| Inf-Vehicle | 4.0 (1.9) | 11.1(8.1) | 3.0(2.2) | 6.9(3.5) * |
| Inf-Ang | −16.7 (7.5) * | −14.8(11.2) * | −5.9(4.6) * | −4.7(3.5) * |
| 1-7 | | | | |
| Inf-Ang | −21.6 (11.5) * | −19.8(9.0) * | −5.3(3.5) * | −6.2(4.4) * |
| Conj. | | | | |

* Significantly different from Inf-Vehicle, p < 0.05
Inf - inflamed animal

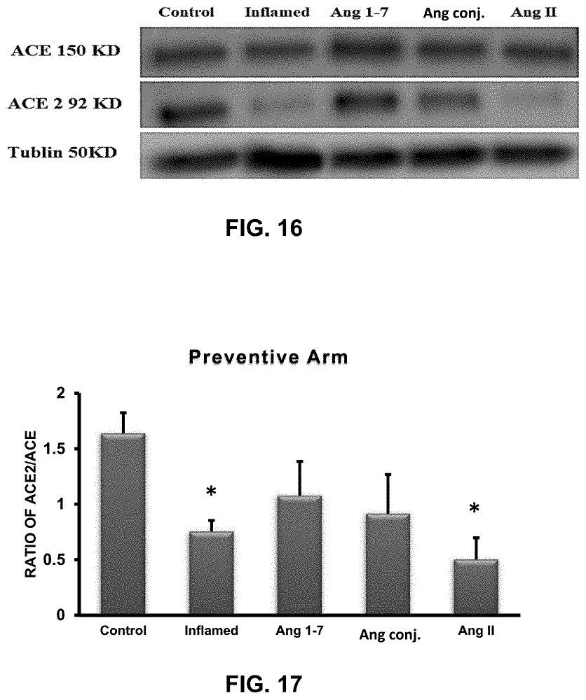

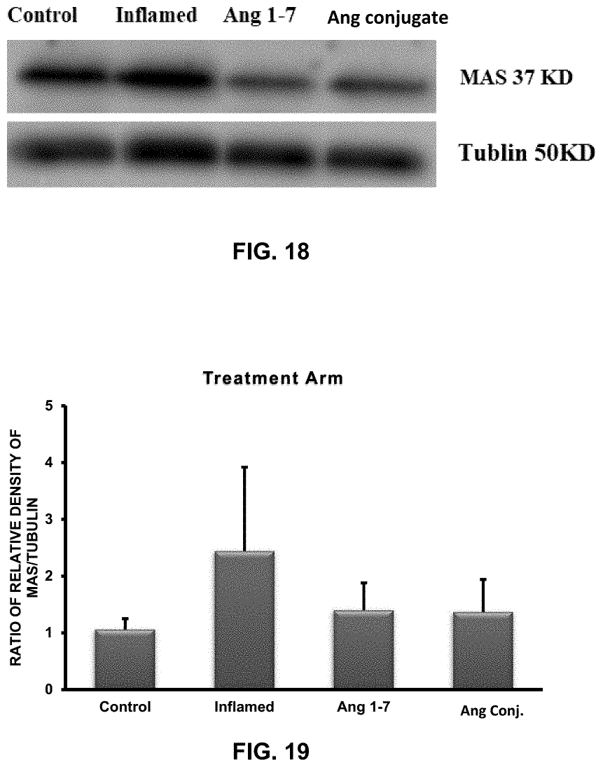

Cardiac ACE2 expression is significantly lowered by AA, giving rise to decreased ACE2/ACE ratio. As indicated in FIG. 16 and FIG. 17, preventive treatment with Ang 1-7 or Ang conjugate restored this balance. In FIG. 18, * indicates significantly different value from the control group (p<0.05).

Cardiac Mas receptor expression is also increased by AA. As shown in FIG. 180 and FIG. 19, treatment after establishment of inflammation with Ang 1-7 or Ang conjugate restored this balance.

### EXAMPLE 5

Three different concentrations of 50, 75, 100 ug/mL of Ang peptide conjugate as described in Example 1 were prepared in water, 20 mM acetate buffer (pH 5.0) and 20 mM of phosphate buffer (pH 7.4) and stored at room temperature (25° C.), refrigerator (4° C.) or freezer (−20° C.). At 1, 3, 7, 10, 15, 30 days; 100 μL of the solutions were sampled and after adding stable Ang II as internal standard samples analyzed by HPLC-UV to monitor the stability of the conjugate over time. Table 3, below, provides the percent of the Ang peptide conjugate remaining in each sample after storage for 4 weeks.

TABLE 3

| Storage condition | 25 ° C. | | 4 ° C. | | −20 ° C. | |
|---|---|---|---|---|---|---|
| Length of storage (day) | 5 | 28 | 5 | 28 | 5 | 28 |
| Acetate buffer (pH 5.5) | 94.3 | 93.3 | 98.4 | 98.0 | 99.8 | 99.5 |
| D. D. Water (pH 6.5) | 90.6 | 64.0 | 95.9 | 92.4 | 99.5 | 99.1 |
| Phosphate buffer (pH 7.4) | 72.8 | 22.6 | 91.9 | 79.2 | 99.2 | 98.9 |

### EXAMPLE 6

In order to evaluate the bone mineral affinity of Ang peptide conjugates, hydroxyapatite (HA) binding in vitro studies were conducted. Briefly, 20 μg of Ang peptide conjugate, as described in Example 1, was mixed with 5 mg of HA powder in 750 μL of binding buffer of various concentrations (double-distilled water, acetate buffer [pH 5.5], 10 mM PBS [pH 7.4], 50 mM PBS [pH 7.4]) and shaken gently at room temperature for 1 hour. Similarly, 20 μg of Ang peptide conjugate in corresponding buffers without HA was used as negative control. After 1 hour, the suspension was centrifuged at 10,000 g for 5 minutes, supernatant was transferred to a fresh tube and the HA pellets were washed three times using 750 μL of above corresponding incubation buffers and centrifuged again to separate the supernatant form the pellet. Supernatants were assayed for unbound drug using fluorescence spectrometer (λEx 215 nm, λEm 305 nm). The percentage of HA binding was calculated as:

(Intensity of control−cumulative intensity of supernatants)/Intensity of control×100%

The results are illustrated in FIG. 20. In FIG. 20, * indicates significantly different binding results (p<0.05) and Ang Conj. refers to the Ang conjugate.

### EXAMPLE 7

Anti-cancer effects of Ang peptide conjugate were examined on a synovial sarcoma mouse model. Tumor-bearing animals were divided into 3 groups and treated with vehicle (Control, n=3), 400 μg/kg, Ang 1-7 (Ang 1-7, n=4) and equivalent dose of Ang peptide conjugate as described in Example 1 (Ang Conj, n=4), twice daily for 3 weeks.

Body weight and tumor size were measured every other day. qPCR and WB assay was performed on tumor tissues to quantify the mRNA and protein levels of ACE2, AT1R, MAS receptor, and caspase-3 as an apoptosis marker.

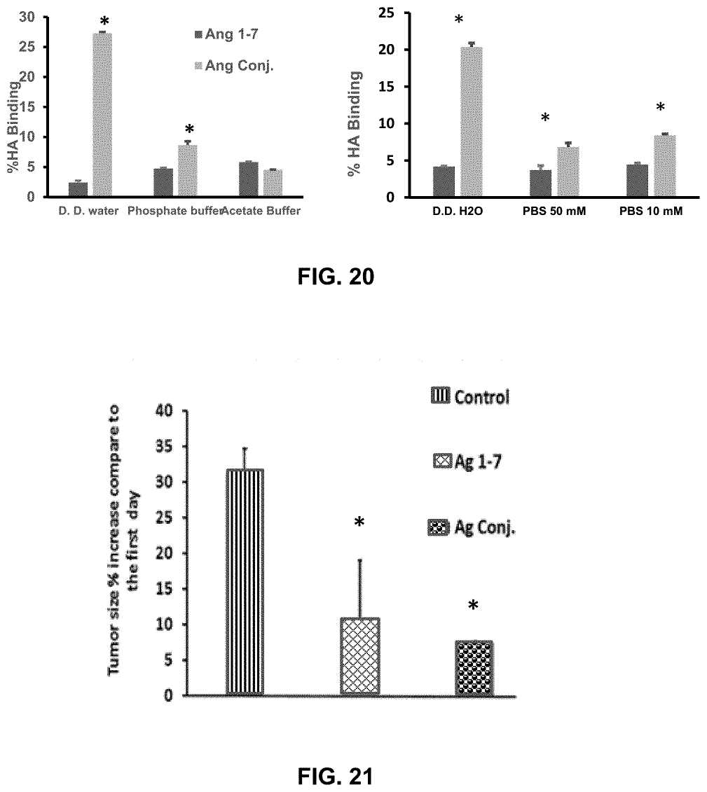

FIG. 21 illustrates the effect of vehicle, Ang 1-7, and Ang conjugate treatment on % tumor size change compared to day one in the osteosarcoma bearing mice. * indicates significantly different from group (p<0.05).

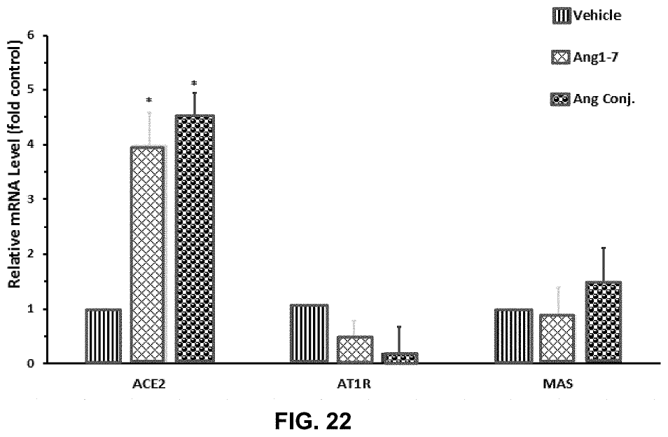

FIG. 22 illustrates the effects of vehicle, Ang 1-7, and Ang conjugate treatment on the RAS components gene expression in tumor tissue from the osteosarcoma bearing mice. Data are presented as relative to the value of control (mean±SD), * p<0.05 vs. control.

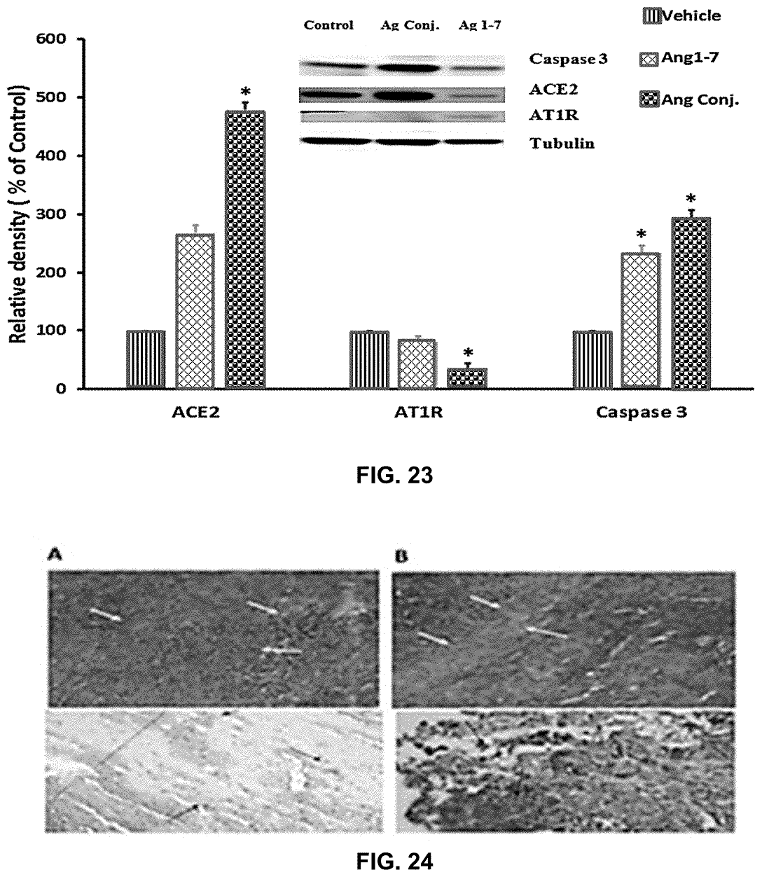

FIG. 23 illustrates effects of vehicle, Ang conjugate, and Ang 1-7 on the RAS and apoptosis maker protein expression in tumor tissue from osteosarcoma-bearing mice. Data are presented as relative to the value of control (mean±SEM), * p<0.05 vs. control.

FIG. 24 presents H & E (upper panel, cell nuclei) and IHC (lower panel, caspase-3 antibody as an apoptosis marker) staining of mouse synovial sarcoma tumor treated with vehicle (left panels) or Ang conjugate (right panels).

While certain embodiments of the disclosed subject matter have been described using specific terms, such description is for illustrative purposes only, and it is to be understood that changes and variations may be made without departing from the spirit or scope of the subject matter.

What is claimed is:

1. A peptide conjugate comprising an Ang peptide, a linking agent comprising a first end and a second end, the linking agent being bonded to a N-terminus of the Ang peptide at the first end of the linking agent, and a targeting moiety bonded to the linking agent at the second end of the polymeric linking agent.

2. The peptide conjugate of claim 1, wherein in the Ang peptide comprises Ang 1-7 (SEQ ID NO: 1) or SEQ ID NO: 2 or an Ang peptide analogue or functional fragment of an Ang 1-7 peptide (SEQ ID NO: 1).

3. The peptide conjugate of claim 1, wherein the linking agent comprises a polymeric linking agent.

4. The peptide conjugate of claim 3, wherein the polymeric linking agent comprises a poly (ethylene glycol).

5. The peptide conjugate of claim 4, wherein the poly (ethylene glycol) is a monodisperse poly (ethylene glycol).

6. The peptide conjugate of claim 3, wherein about 80% or more of the polymeric linking agent has an identical molecular weight.

7. The peptide conjugate of claim 1, wherein the targeting moiety comprises a bone targeting sulfhydryl group.

8. The peptide conjugate of claim 7, wherein the targeting moiety comprises a bisphosphonate.

9. The peptide conjugate of claim 8, wherein the bisphosphonate has a structure of: 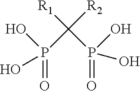
in which R₁ and R₂ are independently selected from hydrogen, substituted or unsubstituted alkyl, substituted or unsubstituted cycloalkyl, substituted or unsubstituted aryl, substituted or unsubstituted heteroaryl, substituted or unsubstituted heteroalicyclic, halo, hydroxy, thiol, alkoxy, thioalkoxy, aryloxy, thioaryloxy, amine, or alkylamine.

10. The peptide conjugate of claim 8, in which R₁ is hydroxy or hydrogen and R₂ is a thioalkyl, alkylamine, or alkoxy including an alkyl chain of from 1 to about 10 carbon atoms in length.

11. A method of forming a peptide conjugate comprising:
forming an Ang peptide according to a solid phase synthesis technique, the peptide formation including protecting N-alpha amino groups of amino acids of the Ang peptide;
reacting a N-terminus of the Ang peptide with a first terminus of a linking agent;
reacting a second terminus of the linking agent with a targeting moiety; and
deprotecting the N-alpha amino groups of amino acids of the Ang peptide.

12. The method of claim 11, wherein the method forms a single isomer of the peptide conjugate at a purity of about 90% or greater.

13. The method of claim 11, wherein the Ang peptide comprises Ang 1-7 (SEQ ID NO: 1) or SEQ ID NO: 2 or an Ang peptide analogue or functional fragment of an Ang 1-7 peptide (SEQ ID NO: 1).

14. The method of claim 11, wherein about linking agent comprises a polymeric linking agent.

15. The method of claim 14, wherein the polymeric linking agent is a monodisperse polymeric linking agent.

16. The method of claim 11, wherein the 7 targeting moiety comprises a bone targeting sulfhydryl group.

17. The method of claim 16, wherein the bisphosphonate has a structure of: 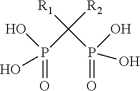
in which R₁ and R₂ are independently selected from hydrogen, substituted or unsubstituted alkyl, substituted or unsubstituted cycloalkyl, substituted or unsubstituted aryl, substituted or unsubstituted heteroaryl, substituted or unsubstituted heteroalicyclic, halo, hydroxy, thiol, alkoxy, thioalkoxy, aryloxy, thioaryloxy, amine, or alkylamine.

18. A method for delivering an Ang peptide to a population of cells comprising contacting an area including the population of cells with a peptide conjugate that comprises an Ang peptide, a linking agent bonded to a N-terminus of the Ang peptide at a first end of the linking agent, and a targeting moiety bonded to the linking agent at a second end of the linking agent.

19. The method of claim 18, wherein the targeting moiety comprises a sulfhydryl group and the area comprises hydroxyapatite.

20. The method of claim 18, wherein the population of cells comprises cancer cells.

21. The method of claim 18, wherein the peptide conjugate exhibits a half-life in the area of about 300 minutes or more.
